(19) United States
(12) Patent Application Publication
(10) Pub. No.: US 2007/0048295 A1
(43) Pub. Date: Mar. 1, 2007

(54) Method for preparing alginate capsules

(75) Inventors: Ming-Ju Chen, Taipei (TW); Kun-Nan Chen, Taipei County (TW); Hsin-Yi Chiu, Taipei (TW); Chin-Wen Lin, Taipei (TW)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Ming-Ju CHEN

(21) Appl. No.: 11/210,081

(22) Filed: Aug. 23, 2005

(51) Int. Cl. A61K 35/74; A61K 38/43; A61K 9/50; A61K 9/16

(52) U.S. Cl. 424/93.45; 424/490; 424/94.1

(57) ABSTRACT

The present invention relates to a method for preparing alginate capsules, including incorporating at least one prebiotic and at least one pancreatic digested protein in the wall of an alginate capsule. The present invention also relates to the alginate capsules prepared by the method.

### BACKGROUND OF THE INVENTION

The present invention relates generally to a method for preparing capsules for oral administration, particularly alginate capsules. The present invention also provides alginate capsules prepared by the method.

Encapsulation is a chemical or mechanical process for enveloping active ingredients in polymeric matrices, providing protection and controlled release of the active ingredients as well as a convenient delivery for the ingredients. Various applications, materials, and techniques for encapsulation have been extensively disclosed. Polymeric matrix encapsulation of microorganisms is a relatively new technology. The most usual hydroxyl polymers used for encapsulating biomaterials are alginate, polyacrylamide, carrageenan, agar, or agarose. Of these, alginate and carrageenan are the only ones which can be manufactured simply in spherical form with encapsulated materials. This is done by ionotropic gelling, i.e., alginate is dropped down into a calcium solution and carrageenan is dropped down into a potassium solution.

As an encapsulating material, calcium alginate is preferred because of its simplicity, non-toxicity, biocompatibility, and low cost (Sheu and Marshall, 1993, J. Food Sci., 54: 557-561). Alginate is a linear heteropolysaccharide of D-mannuronic and L-guluronic acids extracted from various species of algae. The functional properties of alginate as a supporting material are strongly associated with the composition and sequence of L-guluronic and D-mannuronic acids. Divalent cations such as Ca²⁺ bind preferentially to the polymer of L-guluronic acid (Krasaekoopt et al., 2003, Int. Dairy J., 13:3-13). The solubilization of the alginate gel by sequestering of calcium ions and release of the entrapped microorganisms within the digestive tract is another advantage of calcium alginate.

A probiotic can be defined as a living microbial supplement, which can improve the balance of intestinal microorganisms. Good probiotic viability and activity are considered essential for optimal functionality. The survival and multiplication of probiotics in the host strongly affect their probiotic benefits. The probiotic bacteria supplemented in food products should remain metabolically stable and active, surviving passage through the upper digestive tract in large numbers to produce beneficial effects when in the host intestine. Many studies have shown low viability of probiotics in dairy products including yogurt and fermented milk, and protection of the probiotics has been proposed for various dairy fermentations, with encapsulation in hydrocolloidal beads investigated for improving probiotic viability in both the food products and the intestinal tract (Prevost and Divies, 1988, Milchwissenschaft, 43:621-625; Lacroix et al., 1990, Applied Microbiology and Biotechnology, 32:403-408; Champagne et al., 1992, Applied and Environmental Microbiology, 58:1429-1434).

Batich and Vaghefi have disclosed a method for encapsulating Oxalobacter formigenes in alginate or cellulose acetate phthalate (CAP) microcapsules (U.S. Pat. No. 6,242,230). However, Batich's method requires additional post-treatment of the formed microcapsules, including coating the microcapsules with one or more layers of poly-L-lysine (for the alginate capsules) or polyvinylpyridine (for the CAP capsules). Such post-treatment complicates the process and incurs additional expense.

Accordingly, there exists a need to develop a more effective and convenient method of encapsulating drugs, enzymes, microorganisms or other substances, especially probiotic bacteria. The present invention satisfies this need.

As used herein, the symbol “%” or the term “percent” means percent by weight of the particular ingredient or component with which it is used with respect to the volume of the solvent(s) or liquids containing the ingredient or component, unless another meaning is clear from the context in which the symbol or term is used.

### BRIEF SUMMARY OF THE INVENTION

The present invention provides a method for preparing alginate capsules to provide a more effective and convenient method of encapsulating active ingredients.

In accordance with an embodiment of the present invention, there is provided a method for preparing alginate capsules, comprising the steps of:

(a) providing a matrix-forming solution comprising about 1% to about 3% of sodium alginate, up to about 5% of pancreatic digested protein, and up to about 5% of at least one prebiotic;

(b) making droplets of the matrix-forming solution;

(c) introducing the droplets into a calcium chloride solution to form alginate capsules; and

(d) allowing the formed alginate capsules to solidify.

Also in accordance with the present invention, there is provided alginate capsules prepared by a method comprising the steps of:

(a) providing a matrix-forming solution comprising about 1% to about 3% of sodium alginate, up to about 5% of pancreatic digested protein, and up to about 5% of at least one prebiotic;

(b) making droplets of the matrix-forming solution;

(c) introducing the droplets into a calcium chloride solution to form alginate capsules; and

(d) allowing the formed alginate capsules to solidify.

Additional features and advantages of the present invention will be set forth in part in the description which follows, and in part will be apparent from the description, or may be learned by practice of the invention. The features and advantages of the invention will be realized and attained by means of the elements and combinations particularly pointed out in the appended claims.

It is to be understood that all of the foregoing summary, general description and the following detailed description are exemplary and explanatory only and are not restrictive of the invention, as claimed.

The accompanying drawings, which are incorporated in and constitute a part of this specification, illustrate one embodiment of the present invention and together with the description, serve to explain the principles of the invention.

### BRIEF DESCRIPTION OF THE SEVERAL VIEWS OF THE DRAWINGS

Reference will now be made in detail to the present embodiments of the invention, examples of which are illustrated in the accompanying drawings.

FIG. 1 comprises FIGS. 1A and 1B, and shows the survival of the capsules wherein Lactobacillus and Bifidobacterium probiotics, respectively, were encapsulated according to the invention in distilled water for 12 weeks of storage with three different microcapsule formulations.

FIG. 2 comprises FIGS. 2A and 2B, and shows the survival of the capsules wherein Lactobacillus and Bifidobacterium probiotics, respectively, were encapsulated according to the invention in distilled water after 12 weeks of storage and followed by testing in simulated gastric fluid with three different microcapsule formulations.

FIG. 3 comprises FIGS. 3A and 3B and shows the survival of the capsules wherein Lactobacillus and Bifidobacterium probiotics, respectively, were encapsulated according to the invention in distilled water after 12 weeks of storage and followed by testing in simulated bile salts with three different microcapsule formulations.

### DETAILED DESCRIPTION OF THE INVENTION

The present invention provides novel methods for preparing alginate capsules. The alginate capsules prepared according to the present invention have enhanced stability so that they can maintain their structural integrity for a long period of time. Therefore, the alginate capsules of the present invention can provide improved protection for encapsulated active ingredients.

According to the present invention, it is surprisingly found that an incorporation of at least one prebiotic and at least one pancreatic digested protein into the wall of alginate capsules can enhance the strength of the capsules. As used herein, the term “prebiotic” refers to a non-digestible food ingredient that beneficially affects the host by selectively stimulating the growth and/or activity of one or a number of bacteria in the colon. Non-limiting examples of prebiotics include an isomaltooligosaccharide (IMO), a galatooligosaccharide (GOS) and a fructooligosaccharide (FOS), inulin, lactilol, lactulose and pyrodextrin, among others known to those skilled in the art in view of this disclosure, and mixtures thereof. Preferably, the prebiotic used in the present invention is FOS.

As used herein, the term “pancreatic digested protein” refers to the peptide fragments resulting from digesting a protein with pancreatic trypsin. Non-limiting examples of suitable pancreatic digested proteins are casein, gelatin, meat proteins, among others. Preferably, the pancreatic digested protein used in the present invention is pancreatic digested casein. Presently, the more preferred pancreatic digested protein is a product No. 107213 identified as peptone, from pancreatic digested casein, obtained from Merck & Co., Inc., Whitehouse Station, New Jersey, U.S.A.

The method of the present invention comprises the following steps:

(a) providing a matrix-forming solution comprising about 1% to about 3% of sodium alginate, up to about 5% of pancreatic digested protein, and up to about 5% of at least one prebiotic;

(b) making droplets of the matrix-forming solution;

(c) introducing the droplets into a calcium chloride solution to form alginate capsules; and

(d) allowing the formed alginate capsules to solidify.

The method of the present invention is based on the extrusion technique of encapsulation disclosed by Krasaekoopt et al., 2004, International Dairy Journal, 114:737-743, the disclosure of which is hereby incorporated herein by reference. To practice the method of the present invention, a sodium alginate solution is made into droplets by using, for example, a syringe, by which the droplets are introduced into a calcium chloride solution to form alginate capsules. The size of the formed alginate capsules depends on the internal diameter of the needle on the syringe. One can use a needle with a smaller internal diameter to make so-called microcapsules (i.e., capsules with a diameter of less than 5,000 μm).

The method of the present invention is technically distinguished from the prior art by the incorporation of at least one prebiotic and at least one pancreatic digested protein into the wall of alginate capsules. The prebiotic and pancreatic digested protein are added at a concentration of up to about 5% to the sodium alginate solution before it is allowed to react with the calcium chloride solution. The concentration of sodium alginate solution used in the present invention is preferably about 1% to about 3%, although the inventors have found that sodium alginate concentration is not a factor influencing the protection effect of the alginate capsules of the present invention. According to the studies of Sheu and Marshall (1993), both the capsule diameter and protection effect increase when higher sodium alginate concentration is used. However, as can be seen from the results shown in the examples of the present invention, alginate capsules of the present invention formed with about 1% sodium alginate (i.e., the “Optimal Production Model”) and also containing at least one prebiotic and at least one pancreatic digested protein provide better protection than traditional alginate capsules formed with about 3% sodium alginate. Therefore, the addition of at least one prebiotic and at least one peptide pancreatic digested protein does improve the durability of alginate capsules. It is found in the invention that alginate capsules formed with 1% sodium alginate but without at least one prebiotic and at least one pancreatic digested protein are so soft that they are easily broken by an external force. This suggests that at least one prebiotic and at least one pancreatic digested protein promote the formation of alginate capsules.

The concentration of calcium chloride solution used in the present invention is preferably about 0.05M to about 0.3M. It has been reported that when calcium chloride concentration is over 0.02M, it does not substantially influence the strength of the formed alginate capsules. However, it is confirmed in the invention that calcium chloride concentration is not a factor influencing the protection effect of the alginate capsules.

If necessary or desired, the materials for preparing the alginate capsules of the present invention can be sterilized before use. Sterilization can be carried out by any commonly used techniques, such as by an autoclave. However, since the high temperature treatment of sodium alginate will decrease the strength of the formed alginate gel, powdered sodium alginate may be sterilized by UV radiation before adding the powder into an autoclaved solution containing other materials.

The alginate capsules prepared by the method of the present invention can be used to encapsulate various active ingredients including biomaterials and drugs, such as bacteria, viruses, animal or plant cells, algae, fungi, enzymes, peptides, nucleotides, antibiotics, analgesics, antiparasitic drugs, etc. The alginate capsules of the present invention are particularly useful for the administration of viable probiotic bacteria. When probiotic bacteria are encapsulated in the alginate capsules of the present invention and administered orally, they can reach the intestine without harm from the gastric juice and thus exert their probiotic activity in the intestine.

In addition to or instead of administering active ingredients, the alginate capsules of the present invention have other uses. For example, the alginate capsules of the present invention can be manufactured without encapsulating an active ingredient. Such “inactive” capsules can serve as food to replace natural fish eggs for vegetarians.

Preferred embodiments of the present invention will now be described in further detail with reference to the following specific, non-limiting examples.

### EXAMPLES

### Example 1

### Probiotics Culture Conditions

Pure lyophilized cultures of Bifidobacterium longum (CCRC 14605), Lactobacillus casei subsp. rhamnosus (CCRC 12321), B. bifidum (CCRC 11844), and L. acidophilus (CCRC 14079) were purchased from the Culture Collection and Research Center of the Food Industrial Research and Development Institute (Hsinchu, Taiwan, ROC). deMan, Rogosa and Sharp (MRS) and lithium propionate MRS agar (LP-MRS) were used as the selective media for Lactobacillus spp. and Bifidobacterium spp., respectively.

Lactobacillus acidophilus and L. casei were transferred twice in Lactobacilli MRS broth (Difco, France) at 37° C., while Bifidobacterium longum and B. bifidum were transferred twice in MRS broth containing 0.05% L-cysteine hydrochloride (Sigma, USA) in an anaerobic incubator and maintained at 40° C. Cultures were harvested after 24 h by centrifugation (3000×g, 10 min at 4° C.), washed and re-suspended twice in saline solution. The final bacterial counts were adjusted to 10⁹ cells/mL.

### Example 2

### Probiotic Microencapsulation

Probiotic microcapsules were prepared with the extrusion technique of encapsulation disclosed by Krasaekoopt et al., 2004, supra. After washing, 4% of culture concentrate (1% each of Lactobacillus acidophilus, L. casei, Bifidobacterium bifidum and B. longum) was mixed with 50 mL of a sterile solution (autoclaved at 121° C. for 15 min) containing either 1% sodium alginate (Sigma, USA), 3% sodium alginate, or 1% sodium alginate blended with 1% pancreatic digested casein, Cheng-Fung Co., Taiwan) and 3% FOS (Cheng-Fung Co., Taiwan) (designated herein as the “Optimal Production Model”). The thus obtained cell suspensions were injected through a 0.11 mm needle into sterile 0.1M CaCl₂. The microcapsules that formed were approximately 0.5 mm in diameter, and were allowed to stand for 1 hr for gelification, and then rinsed with, and subsequently kept in, sterile 0.1% peptone solution at 4° C.

### Example 3

### Determination of Probiotic Viability

To determine the probiotic viability count, the entrapped probiotics were released from the microcapsules according to the method of Sheu and Marshall, 1993, Journal of Food Science, 54:557-561, the disclosure of which is hereby incorporated herein by reference. One gram of the microcapsules was re-suspended in 9 mL of phosphate buffer (0.1 M, pH 7.0) followed by homogenization in a Seward Stomacher® Model 400C lab blender (Brinkmann Instruments, Inc., Westbury, N.Y., USA) for 15 min. The suitability of the media was tested by plating decimal dilutions of the probiotic cultures. Thus, a 1-g sample was decimally diluted into sterile peptone water (0.1%), and then 0.1-mL aliquot dilutions were plated onto the different media, in triplicate. Plates of MRS agar were incubated aerobically for 72 h at 37° C. to inhibit bifidobacteria. Plates of LP-MRS agar (GasPak System; Oxoid Unipath Ltd, Basingstoke, Hampshire, England) were incubated anaerobically (72 h at 37° C.) before enumeration of the bifidobacteria. The population, in colony-forming units (CFU), and the characteristics of the colonies were recorded for each medium.

### Example 4

### Survival of Microencapsulated Probiotics in Milk

Mixed probiotics (1% each of Lactobacillus acidophilus, L. casei, Bifidobacterium bifidum and B. longum) were either added as free cells to the milk (3.5% milk fat, National Taiwan University, Taipei, Taiwan) or as entrapped cells in microcapsules. The samples were stored at 4° C. for 16 days and the probiotic viability was determined.

Survival of probiotics entrapped in the microcapsules of the Optimal Production Model immersed in refrigerated milk for 16 days was significantly improved over that of free cells as shown in the following table. Similar results have been reported previously by others for probiotic microencapsulated in alginate microcapsules.

| Probiotics | | | | | |
|---|---|---|---|---|---|
| culture | Storage period (days) | | | | |
| Log CFU/g | 0 | 4 | 8 | 12 | 16 |
| Free L^{a} | 8.57 ± 0.16 | 8.42 ± 0.13 | 8.02 ± 0.07 | 7.51 ± 0.14 | 6.57 ± 0.12 |
| Free B^{b} | 8.11 ± 0.09 | 7.83 ± 0.12 | 6.82 ± 0.13 | 6.11 ± 0.09 | 5.89 ± 0.14 |
| M^{c} L | 8.12 ± 0.11 | 8.15 ± 0.21 | 8.10 ± 0.17 | 8.10 ± 0.18 | 8.03 ± 0.20 |
| M B | 8.01 ± 0.18 | 8.03 ± 0.09 | 7.98 ± 0.15 | 7.98 ± 0.11 | 7.90 ± 0.14 |

^{a}L: L. acidophilus + L. casei
^{b}B: B. longum + B. bifidum
^{c}M: Microencapsulated

### Example 5

### Storage Test

In order to understand the effect of the use of prebiotics in forming the alginate capsule on the microencapsulated probiotics during storage, the viable cell counts for the encapsulated organisms were determined. In addition to the microcapsules of the Optimal Production Model, microcapsules with 1% and 3% alginate, without the prebiotic or pancreatic digested protein, were also tested. The three kinds of microcapsules were immersed in aseptic water and stored at 4° C. for 3 months, with the survival of the encapsulated probiotics determined every two weeks.

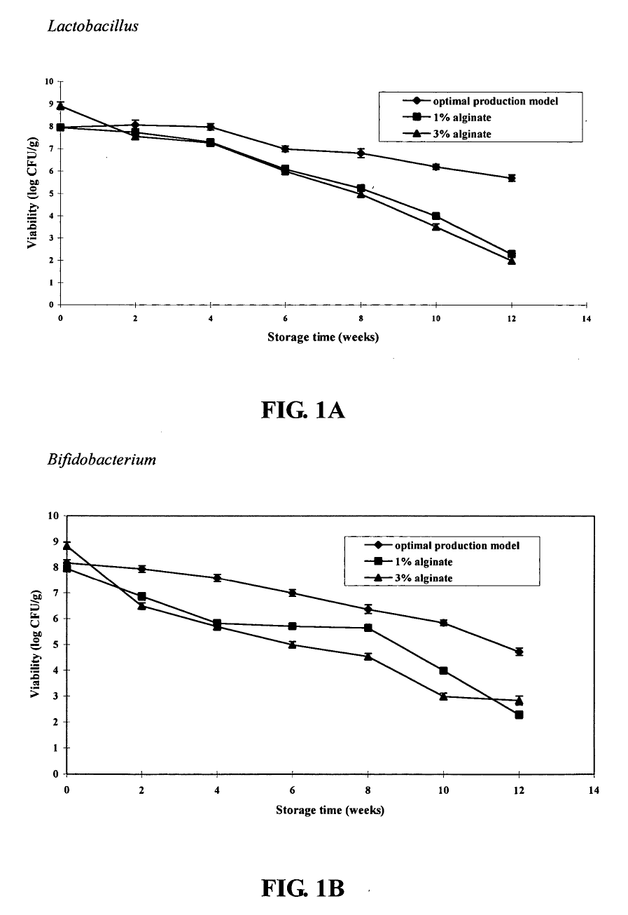

The results of the probiotic counts showed that, as might be expected, viability decreased with increasing storage period for all three microcapsule formulations (FIG. 1). The probiotic counts for the microcapsules of the Optimal Production Model were still 10⁵-10⁶ CFU/g after the 12-week storage in contrast to just 10²-10³ CFU/g for those without the prebiotics and without a pancreatic digested protein used to make the capsules. Thus, blending of prebiotics and pancreatic digested protein in the coating materials resulted in better protection for the encapsulated organisms during storage, relative to the microcapsules without the prebiotic and pancreatic digested protein capsule variants.

### Example 6

### Survival of Encapsulated Probiotics in Simulated Gastric Fluid Test (SGFT) and Bile-Salt Conditions

As described in Example 5, the three kinds of microcapsules were immersed in aseptic water and stored at 4° C. for 3 months, with the survival of the encapsulated probiotics in simulated gastric fluid and bile salt treatments determined every two weeks.

Resistance to simulated gastric fluid was determined by adding 1 g of the microencapsulated bacteria into flasks containing 10 mL of the simulated gastric juice, which consisted of 0.3% pepsin (Sigma, USA) and 0.5% sodium chloride (Nakalai, Japan) adjusted to pH 2.0 with 1 N HCl. Resistance to bile salts was determined by adding microencapsulated bacteria to the bile-salt solution, which consisted of 2% ox gall powder (Sigma, USA). Both resistance treatments took place in agitated flasks (100 rpm) at 25° C. for 1 hr.

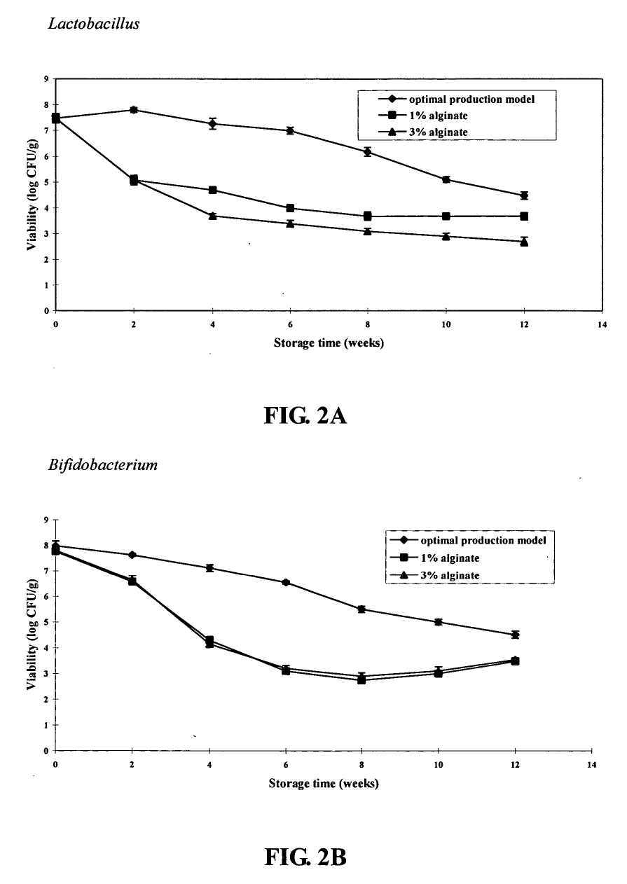

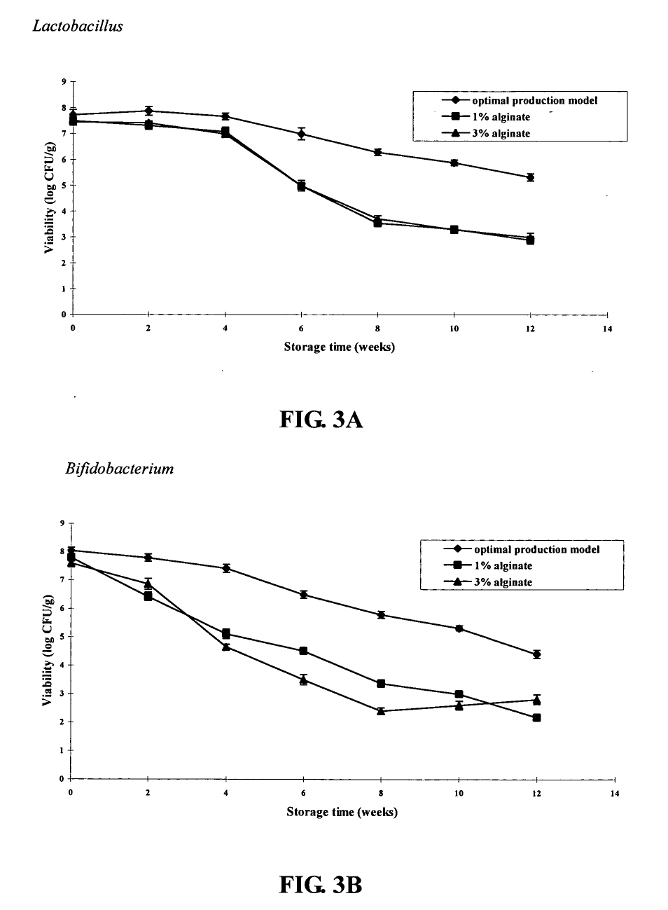

The effects of encapsulating materials and sodium alginate concentrations on the viability of Lactobacillus spp. and Bifidobacterium spp. under simulated gastric fluid and bile salt conditions after storage are shown in FIGS. 2 and 3, respectively. The probiotic and pancreatic digested protein microcapsules of the Optimal Production Model produced the highest viable cell counts for both Lactobacillus spp. and Bifidobacterium spp. under the simulated gastric fluid test after storage than those microcapsules without prebiotics and pancreatic digested proteins. Probiotic counts for the microcapsules of the Optimal Production Model remained at 10⁵-10⁶ CFU/g after 8 weeks of storage, compared to only 10²-10³ CFU/g survival for the 1% and 3% alginate counterparts without prebiotic and without pancreatic digested protein. However, both Lactobacillus spp. and Bifidobacterium spp. showed a decrease of 1 log cycle as compared to the initial cell counts (FIG. 2) without significant difference among the three treatments. The survival of microorganisms is affected by low pH of the environment. Our results demonstrated that microencapsulation with calcium alginate with at least one prebiotic and at least one pancreatic digested protein, rather than just alginate alone, could provide a good protection for probiotics under simulated gastric fluid testing.

The results of our preliminary test and other studies showed that probiotics had higher tolerance to acid than to bile salts. In this sense, it is generally considered necessary to evaluate the ability of potentially microencapsulated probiotic bacteria to resist the effect of bile salts. Probiotic counts for the microcapsules of Optimal Production Model remained at 10⁵-10⁶ CFU/g after 8 weeks of storage, compared to only 10²-10³ CFU/g survival for the 1% and 3% alginate counterparts, which is similar to the results of the simulated gastric fluid test (as shown in FIG. 3). Both Lactobacillus spp. and Bifidobacterium spp. showed a decrease of 1 log cycle as compared to the initial cell count (FIG. 3).

In describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily, in view of the present disclosure, appreciate that the sequences may be varied and still remain within the spirit and scope of the present invention.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

We claim:

1. A method for preparing alginate capsules, comprising the steps of:
(a) providing a matrix-forming solution comprising about 1% to about 3% of sodium alginate, up to 5% of pancreatic digested protein, and up to about 5% of at least one prebiotic;
(b) making droplets of the matrix-forming solution;
(c) introducing the droplets into a calcium chloride solution to form alginate capsules; and
(d) allowing the formed alginate capsules to solidify.

2. The method according to claim 1, wherein the prebiotic is selected from the group consisting of an isomaltooligosaccharide (IMO), a galatooligosaccharide (GOS), a fructooligosaccharide (FOS), inulin, lactilol, lactulose, pyrodextrin and mixtures thereof.

3. The method according to claim 1, wherein the prebiotic is fructooligosaccharide (FOS).

4. The method according to claim 1, wherein the pancreatic digested protein is pancreatic digested casein.

5. The method according to claim 2, wherein the pancreatic digested protein is pancreatic digested casein.

6. The method according to claim 3, wherein the pancreatic digested protein is pancreatic digested casein.

7. The method according to claim 1, wherein the matrix-forming solution in step (a) is prepared by adding UV-sterilized sodium alginate powder into an autoclaved solution containing pancreatic digested protein and at least one prebiotic.

8. The method according to claim 6, wherein the matrix-forming solution in step (a) is prepared by adding UV-sterilized sodium alginate powder into an autoclaved solution containing pancreatic digested protein and at least one prebiotic.

9. The method according to claim 1, further including the step of adding one or more active ingredients to be encapsulated into the matrix-forming solution in step (a).

10. The method according to claim 6, further including the step of adding one or more active ingredients to be encapsulated into the matrix-forming solution in step (a).

11. The method according to claim 9, wherein the active ingredient to be encapsulated is a biomaterial.

12. The method according to claim 10, wherein the active ingredient to be encapsulated is a biomaterial.

13. The method according to claim 11, wherein the biomaterial is selected from the group consisting of at least one bacterium, virus, animal or plant cell, alga, fungus, enzyme, peptide, and nucleotide.

14. The method according to claim 12, wherein the biomaterial is selected from the group consisting of at least one bacterium, virus, animal or plant cell, alga, fungus, enzyme, peptide, and nucleotide.

15. The method according to claim 13, wherein the biomaterial is a bacterium.

16. The method according to claim 14, wherein the biomaterial is a bacterium.

17. The method according to claim 15, wherein the bacterium is a probiotic bacterium.

18. The method according to claim 16, wherein the bacterium is a probiotic bacterium.

19. The method according to claim 17, wherein the probiotic bacterium is selected from the group consisting of Lactobacillus acidophilus, Lactobacillus casei, Bifidobacterium bifidum and Bifidobacterium longum, and mixtures thereof.

20. The method according to claim 18, wherein the probiotic bacterium is selected from the group consisting of Lactobacillus acidophilus, Lactobacillus casei, Bifidobacterium bifidum and Bifidobacterium longum, and mixtures thereof.

21. The method according to claim 13, wherein the biomaterial is an enzyme.

22. The method according to claim 14, wherein the biomaterial is an enzyme.

23. The method according to claim 9, wherein the active ingredient to be encapsulated is a drug.

24. The method according to claim 10, wherein the active ingredient to be encapsulated is a drug.

25. The method according to claim 1, wherein the calcium chloride solution used in step (b) has a concentration of about 0.05M to about 0.3M.

26. The method according to claim 6, wherein the calcium chloride solution used in step (b) has a concentration of about 0.05M to about 0.3M.

27. The method according to claim 25, wherein the calcium chloride solution used in step (b) has a concentration of about 0.1 M.

28. The method according to claim 26, wherein the calcium chloride solution used in step (b) has a concentration of about 0.1 M.

29. The method according to claim 1, wherein the matrix-forming solution in step (a) includes about 1% to about 3% of sodium alginate, about 1% of pancreatic digested casein, and about 3% of fructooligosaccharide.

30. An alginate capsule prepared by the method of claim 1.

31. The alginate capsule according to claim 30, wherein the alginate capsule includes one or more encapsulated active ingredients.

32. The alginate capsule according to claim 31, wherein the encapsulated active ingredient is a biomaterial.

33. The alginate capsule according to claim 32, wherein the biomaterial is selected from the group consisting of at least one bacterium, virus, animal or plant cell, alga, fungus, enzyme, peptide, and nucleotide.

34. The alginate capsule according to claim 33, wherein the biomaterial is a bacterium.

35. The alginate capsule according to claim 34, wherein the bacterium is a probiotic bacterium.

36. The alginate capsule according to claim 31, wherein the probiotic bacterium is selected from the group consisting of Lactobacillus acidophilus, Lactobacillus casei, Bifidobacterium bifidum and Bifidobacterium longum.

37. The alginate capsule according to claim 6, wherein the alginate capsule includes one or more encapsulated active ingredients.

38. The alginate capsule according to claim 37, wherein the encapsulated active ingredient is a biomaterial.

39. The alginate capsule according to claim 38, wherein the biomaterial is selected from the group consisting of at least one bacterium, virus, animal or plant cell, alga, fungus, enzyme, peptide, and nucleotide.

40. The alginate capsule according to claim 39, wherein the biomaterial is a bacterium.

41. The alginate capsule according to claim 40, wherein the bacterium is a probiotic bacterium.

42. The alginate capsule according to claim 41, wherein the probiotic bacterium is viable.

43. The alginate capsule according to claim 42, wherein the probiotic bacterium is selected from the group consisting of Lactobacillus acidophilus, Lactobacillus casei, Bifidobacterium bifidum and Bifidobacterium longum.
